(19) United States
(12) Patent Application Publication
(10) Pub. No.: US 2023/0382921 A1
(43) Pub. Date: Nov. 30, 2023

(54) Amorphous Form of Isoquinoline Derivative

(71) Applicant: vTv Therapeutics LLC, High Point, NC (US)

(72) Inventors: Dharma Rao POLISETTI, High Point, NC (US); Thomas Scott YOKUM, Greensboro, NC (US); Yuanqiang SUN, Solon, OH (US); James C. QUADA, Jr., High Point, NC (US)

(21) Appl. No.: 18/245,978

(22) PCT Filed: Sep. 17, 2021
(86) PCT No.: PCT/US2021/050841

§ 371 Date: Mar. 20, 2023

Related U.S. Application Data
(60) Provisional application No. 63/080,985, filed on Sep. 21, 2020.

(51) Int. Cl. C07D 491/056

(52) U.S. Cl. CPC C07D 491/056

(57) ABSTRACT

The present invention provides an amorphous form of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid dihydrochloride salt, pharmaceutical compositions comprising the same, methods of preparation and use thereof in treating conditions, such as diabetes, where modulation of the human GLP-1 receptor is beneficial.

### TECHNICAL FIELD

The present invention provides an amorphous form of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid dihydrochloride salt, pharmaceutical compositions comprising the same, methods of preparation and use thereof in treating conditions, such as diabetes, where modulation of the human GLP-1 receptor is beneficial.

### BACKGROUND OF THE INVENTION

Diabetes mellitus type 2 (type 2 diabetes) is a chronic metabolic disorder characterized by a number of symptoms, including, but not limited to, elevated blood-glucose levels, insulin resistance, impaired insulin secretion, and hyperglycemia. Symptoms associated with type 2 diabetes tend to manifest themselves gradually and progressively, becoming worse and greater in number as the disease progresses. If not treated well, type 2 diabetes eventually can lead to heart disease, stroke, blindness (due to diabetic retinopathy), kidney failure, and poor blood circulation to the limbs (which can result in the need to amputate limbs, such as feet and toes, that no longer benefit from sufficient circulation). Type 2 diabetes and its related disorders, such as obesity, pose a major public health problem throughout the world.

The causes of type 2 diabetes are multifactorial in nature and treatment regimens vary. In many cases, type 2 diabetes may be managed by maintaining a normal weight, exercising regularly, and eating properly. But such measures are often insufficient, and antidiabetic medications, such as metformin, are often prescribed. But metformin therapy often fails to affect disease progression in a clinically meaningful way.

Glucagon-like peptide 1 (GLP-1) analogs and glucagon-like peptide 1 receptor (GLP-1R) agonists are a class of therapies that have shown particular promise in treating diabetes and other disorders associated with the dysregulation of glucose metabolism. Because peptides, may lack sufficient oral bioavailability for consideration as oral drug agents, small molecule non-peptide modulators of GLP-1R with oral bioavailability are being developed, such as one or more of the compounds disclosed in International Publication Nos. WO 2009/111700 and WO 2010/114824.

With any small molecule drug, the compound may exist in various solid state forms that possess physical properties that differ from and may be advantageous over those of other solid state forms. These include, packing properties such as molar volume, density and hygroscopicity; thermodynamic properties such as melting temperature, vapor pressure and solubility; kinetic properties such as dissolution rate and stability under various storage conditions; surface properties such as surface area, wettability, interfacial tension and shape; mechanical properties such as hardness, tensile strength, compatibility, handling, flow and blend; and filtration properties. Variations in any one of these properties may affect the chemical and pharmaceutical processing of a compound as well as its bioavailability and may often render the new form advantageous for pharmaceutical and medical use.

There remains an unmet need for additional solid state forms of any compound in development having good physiochemical properties, desirable bioavailability, and advantageous pharmaceutical parameters.

### SUMMARY OF THE INVENTION

(S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid (“OAD2”) is a small molecule, non-peptide glucagon-like peptide 1 (GLP-1) receptor agonist that is in development to treat diabetes and other related indications. OAD2 is disclosed in International publication WO 2010/114824, and the OAD2 dihydrochloride has the following chemical structure:

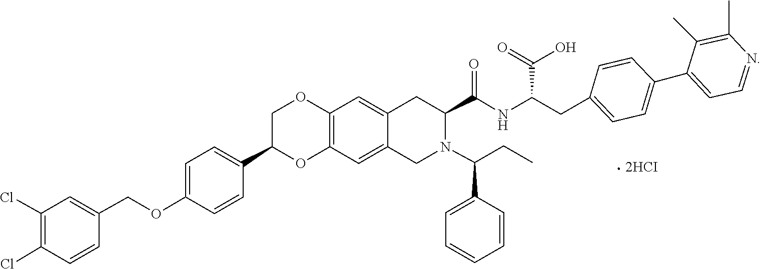 [SMILES: CC[C@@H](c1ccccc1)N1Cc2cc3c(cc2C[C@H]1C(=O)N[C@@H](Cc1ccc(-c2ccnc(C)c2C)cc1)C(=O)O)OC[C@H](c1ccc(OCc2ccc(Cl)c(Cl)c2)cc1)O3]

The present invention provides an amorphous form of OAD2 dihydrochloride, pharmaceutical compositions comprising the same, methods for its preparation, and use thereof in treating conditions, such as diabetes, where modulation of the human GLP-1 receptor is beneficial.

### BRIEF DESCRIPTION OF THE FIGURES

FIG. 1 illustrates a characteristic X-ray powder diffraction pattern (“XRPD”) of an amorphous form of OAD2 dihydrochloride.

FIG. 2 illustrates a characteristic Differential Scanning Calorimetry (“DSC”) profile of an amorphous form of OAD2 dihydrochloride.

FIG. 3 illustrates a characteristic Thermogravimetric Analysis (“TGA”) profile of an amorphous form of OAD2 dihydrochloride.

FIG. 4 illustrates a characteristic Fourier-Transformed Infrared (“FTIR”) profile of an amorphous form of OAD2 dihydrochloride.

FIG. 5 illustrates a characteristic XRPD pattern of another sample of an amorphous form of OAD2 dihydrochloride.

FIG. 6 illustrates a characteristic DSC of another sample of an amorphous form of OAD2 dihydrochloride.

FIG. 7 illustrates a characteristic TGA of another sample of an amorphous form of OAD2 dihydrochloride.

### DETAILED DESCRIPTION OF THE INVENTION

### Definitions

In the invention, “amorphous” or “amorphous form” of a solid may be identified by the characterization through X-ray powder diffraction pattern or other means showing the absence of long-range periodic atomic ordering.

The term “therapeutically effective amount” is used herein to denote the amount of OAD2 that will elicit the therapeutic response of a subject that is being sought. In an embodiment, the therapeutic response may be agonizing the GLP-1 receptor.

### Aspects of Invention

### Amorphous Form of OAD2 Dihydrochloride

In one aspect, the present invention provides an amorphous form of OAD2 dihydrochloride.

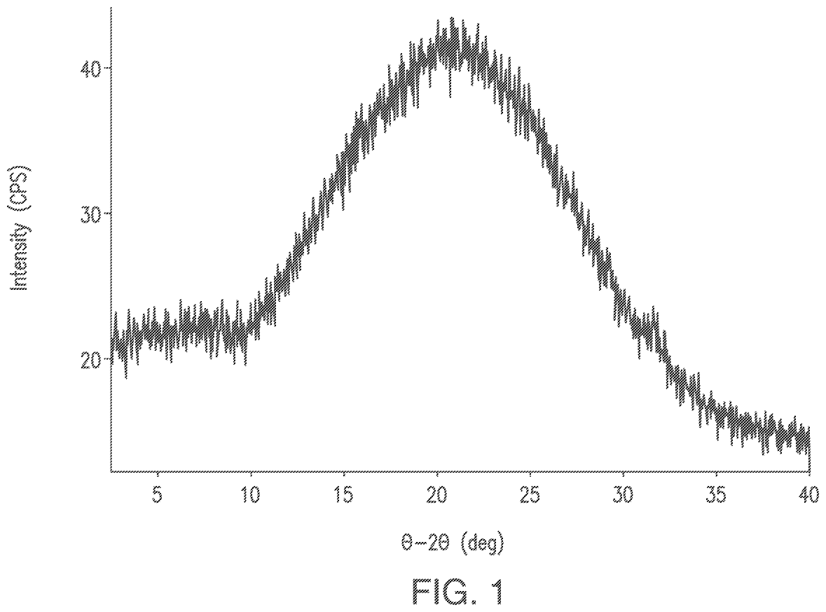

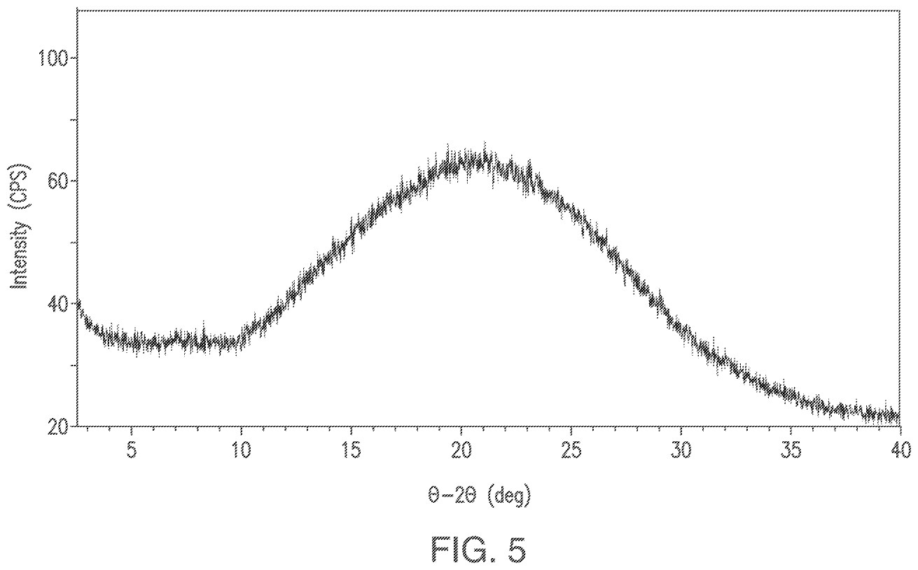

In an embodiment, the amorphous form of OAD2 dihydrochloride is characterized by an XRPD pattern substantially as shown in FIG. 1 or FIG. 5.

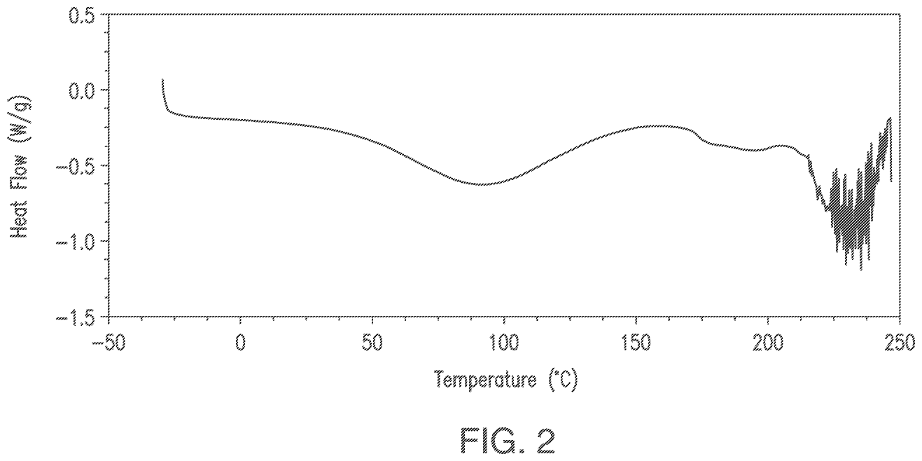

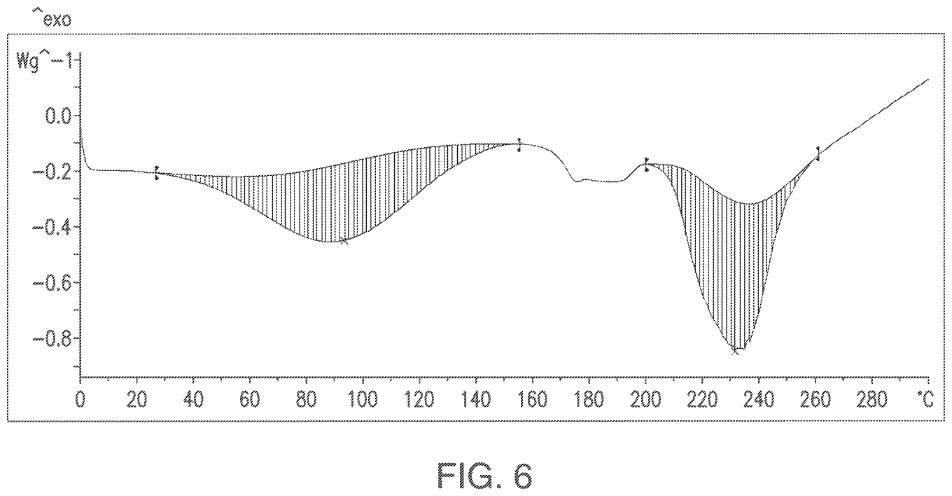

In another embodiment, the amorphous form of OAD2 dihydrochloride is characterized by a DSC profile substantially as shown in FIG. 2 or FIG. 6. In a further embodiment, the amorphous form of OAD2 dihydrochloride is characterized by a DSC profile having a first endothermic peak between 85-105° C. In a further embodiment, the DSC profile has a first endothermic peak between at 90±2° C. optionally with peak onset between 25-40° C. and peak endset between 145-160° C. In a further embodiment, the DSC profile has a broad endothermic peak between 170 and 210° C. In a further embodiment, the DSC profile has an endothermic peak between 210 and 265° C. or between 225 and 235° C.

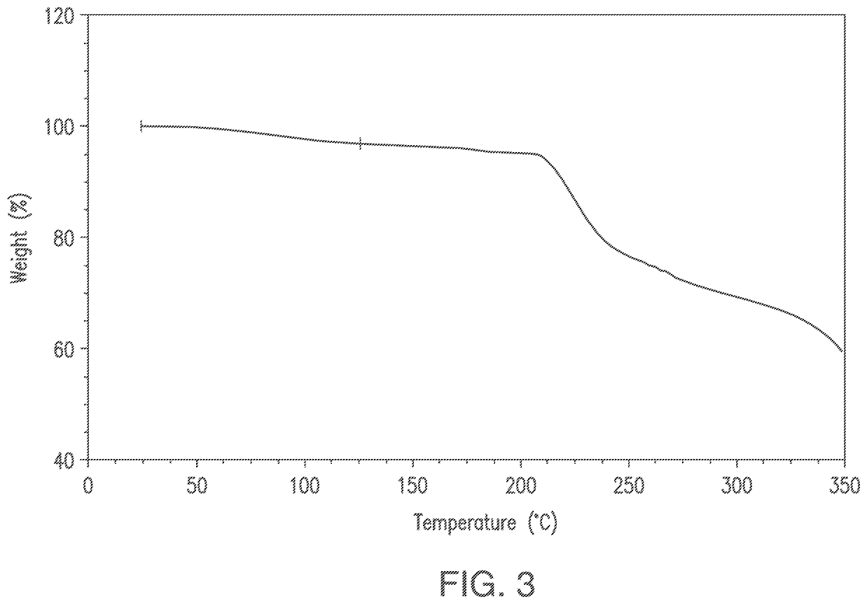

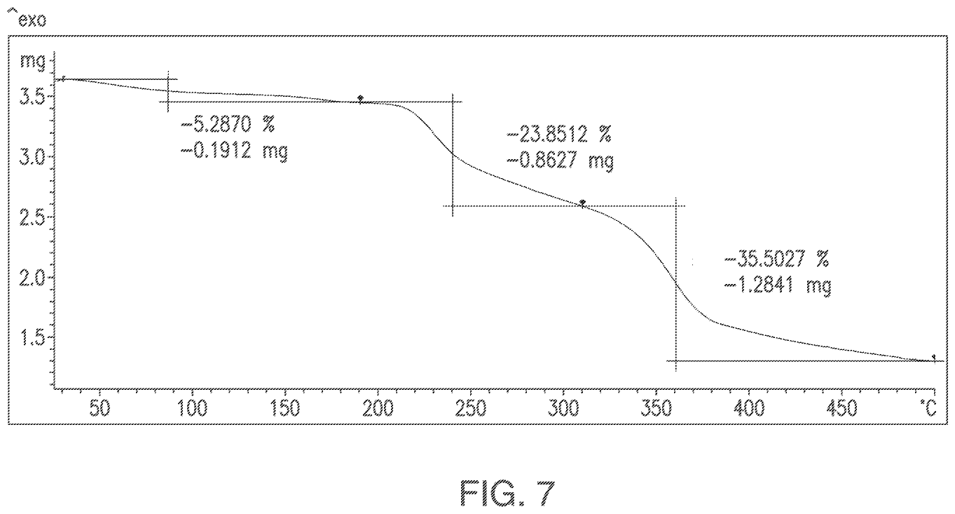

In another embodiment, the amorphous form of OAD2 dihydrochloride is characterized by a TGA profile substantially as shown in FIG. 3 or FIG. 7. In a further embodiment, the amorphous form of OAD2 dihydrochloride is characterized by a TGA profile having a weight loss of between 2-4% from 25 to 125° C. In a further embodiment, the amorphous form of OAD2 dihydrochloride is characterized by a TGA profile having a weight loss of between 22 to 28% from 190 to 310° C.

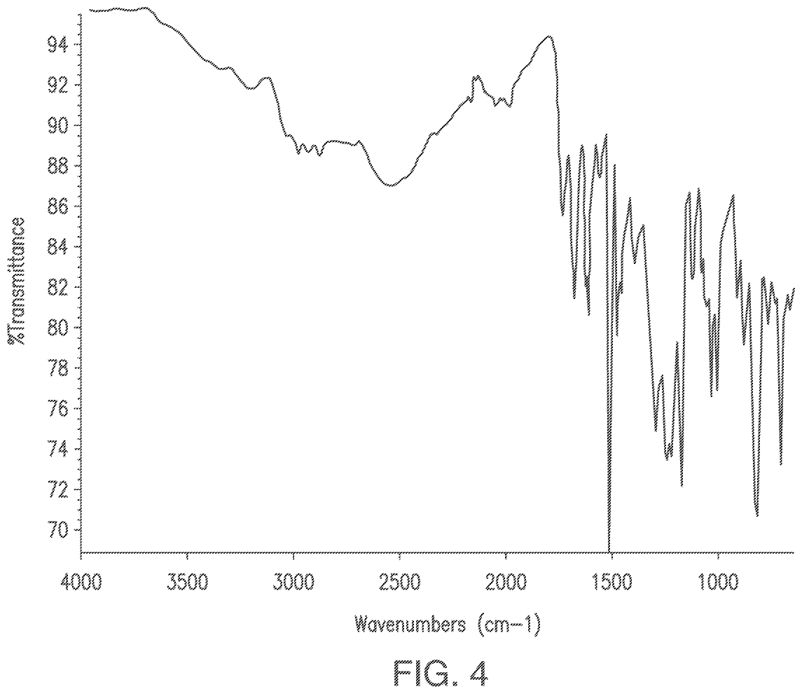

In another embodiment, the amorphous form of OAD2 dihydrochloride is characterized by an IR profile substantially as shown in FIG. 4. In a further embodiment, the amorphous form of OAD2 dihydrochloride is characterized by IR peaks at 1731±2 cm⁻¹, 1625±2 cm⁻¹, 1612±2 cm⁻¹, and 1509±2 cm⁻¹. In a further embodiment, the amorphous form of OAD2 dihydrochloride is characterized by IR peaks at 1731±2 cm⁻¹, 1662±2 cm⁻¹, 1625±2 cm⁻¹, 1612±2 cm⁻¹, 1509±2 cm⁻¹, 1292±2 cm⁻¹, 764±2 cm⁻¹, and 668±2 cm⁻¹.

TABLE 1
Peak Positions in FIG. 4 of Amorphous Sample
of OAD2 Dihydrochloride Salt
Position
(cm⁻¹)

3361
3207
2977
2931
2876
1731
1662
1625
1612
1562
1509
1473
1458
1393
1292
1241
1220
1171
1123
1079
1050
1029
1003
877
818
764
703
668

In another embodiment, the present invention provides an amorphous form of OAD2 dihydrochloride characterized by any combination of at least two of the embodiments described in the previous paragraphs. For example, an amorphous form of OAD2 dihydrochloride may be characterized by any combination of at least two of the following features 1 to 4:
1) an XRPD profile substantially as shown in FIG. 1 or FIG. 5;
2) a DSC profile substantially as shown in FIG. 2 or FIG. 6;
3) a TGA profile substantially as shown in FIG. 3 or FIG. 7; and
4) an IR profile substantially as shown in FIG. 4.

In another embodiment, the amorphous form of OAD2 dihydrochloride is characterized by a solubility in 0.1 N aqueous HCl of greater than 51 mg/mL or a solubility in a buffered aqueous solution at pH 7 of 0.09 mg/mL.

In another embodiment, the amorphous form of OAD2 dihydrochloride is substantially free of other polymorphic forms. In a further embodiment, the amorphous form of OAD2 dihydrochloride has polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% by weight. In a further embodiment, the amorphous form of OAD2 dihydrochloride has less than 10%, or less than 5%, or less than 1% by weight of a solid form of OAD2 dihydrochloride having long-range molecular order such as crystalline material.

### Methods of Preparation

According to another aspect, the present invention provides methods or processes for preparing an amorphous form of OAD2 dihydrochloride.

In one embodiment, an amorphous form of OAD2 dihydrochloride may be prepared by: i) adding an amount of OAD2 sodium salt or OAD2 monohydrochloride salt to a suitable solvent or solvent system to form a mixture, and ii) adding a sufficient amount of an aqueous solution of HCl to the mixture to precipitate OAD2 dihydrochloride. The solvent may then be removed by evaporation under reduced pressure and/or elevated temperature (25-50° C.). The suitable solvent system may be one where the OAD2 sodium salt or OAD2 monohydrochloride salt will dissolve or will form a slurry. Suitable solvent or solvent systems may include i) aqueous acetic acid (10-80%), ii) glacial acetic acid, iii) mixture of acetic acid and 1-5 molar HCl, iv) tetrahydrofuran and 1-5 molar HCl, and v) acetone and 1-5 molar HCl. The aqueous solution of HCl used in step ii) may be between 1 N to 5 N HCl or between 1 M to 5 M HCl. Further the aqueous solution of HCl used in step ii) may be the same or higher concentration of HCl used in step i).

In another embodiment, an amorphous form of OAD2 dihydrochloride may be prepared by: i) adding OAD2 sodium salt to acetone and forming a slurry, and ii) treating the slurry with an aqueous solution of HCl in an amount sufficient to form solid OAD2 dihydrochloride. In an embodiment, the amount of HCl is at least a 3 fold molar excess relative to the molar amount of OAD2 sodium salt in the slurry. In an embodiment, the aqueous solution of HCl may be 4 N HCl. Following addition of the aqueous solution of HCl, the solvent may then be removed, and the collected solid washed with water and dried under reduced pressure and/or at a temperature between 25 and 50° C.

In another embodiment, an amorphous form of OAD2 dihydrochloride may be prepared by: i) dissolving OAD2 monohydrochloride salt in an aqueous solution of acetic acid and HCl, and ii) adding sufficient amount of aqueous HCl to the aqueous solution to precipitate OAD2 dihydrochloride. The resulting solids may be collected by filtration, washed with aqueous HCl, and dried under reduced pressure and/or at a temperature between 25 and 50° C. In an embodiment, the first step may comprise the initial step of combing OAD2 monohydrochloride salt and acetic acid in a weight to weight ratio of 1:2. In a further embodiment, the first step may comprise combining 5 N HCl.

In another embodiment, an amorphous form of OAD2 dihydrochloride salt may be prepared according to a method that comprises the following steps: 1) dissolving free alkali OAD2 or OAD2 in a mixture comprising an organic solvent and an aqueous hydrochloric acid solution, 2) adding water to the mixture, and 3) adding an aqueous hydrochloric acid solution to the mixture. The resulting solids may be collected by filtration and dried. As a specific embodiment, the organic solvent is miscible with water. In another embodiment, the solvent is one or more selected from tetrahydrofuran, acetone, and acetic acid. In another specific embodiment, the concentration of the hydrochloric acid solution is 2 to 6 mol/L, or 3 to 4 mol/L.

### Pharmaceutical Compositions

The invention also provides a pharmaceutical composition comprising an amorphous form of OAD2 dihydrochloride and a pharmaceutically acceptable carrier.

In an embodiment, the pharmaceutical composition comprises a therapeutically effective amount of an amorphous form of OAD2 dihydrochloride.

In another embodiment, the amorphous form of OAD2 dihydrochloride may be administered orally, and the pharmaceutical compositions may be formulated for oral administration such as tablets (including e.g. film-coated tablets, sublingual tablets and orally disintegrating tablets), powders, granules, dragees, pellets, pills, capsules (including soft capsules) and the like. Each possibility is a separate embodiment of the invention.

Pharmacologically acceptable carriers that may be used in the context of the present invention include various organic or inorganic carriers including, but not limited to excipients, lubricants, binders, disintegrants, water-soluble polymers and basic inorganic salts. The pharmaceutical compositions of the present invention may further include additives such as, but not limited to, preservatives, anti-oxidants, coloring agents, sweetening agents, souring agents, bubbling agents and flavorings.

The pharmaceutical composition may comprise an amount of an amorphous form of OAD2 dihydrochloride between 1 mg and 1000 mg, or between 25 mg to 200 mg, or 25 to 75 mg, or 50 to 100 mg, or 75 to 125 mg, or 75 mg to 150 mg, or 100 mg to 150 mg, or 125 to 175 mg, or 150 mg to 200 mg, or 25 mg, or 50 mg, or 75 mg, or 100 mg, or 125 mg, or 150 mg.

In another embodiment, the present invention provides a pharmaceutical composition comprising an amorphous form of OAD2 dihydrochloride in combination with one or more other active ingredients, for example other anti-diabetic drug(s), within a single pharmaceutical composition.

### Therapeutic Uses

In another aspect, the invention also provides methods of treatment comprising administering to a human in need thereof a therapeutically effective amount of an amorphous form of OAD2 dihydrochloride.

The methods of treatment may useful to treat a disorder or condition where activation of the GLP-1 receptor is beneficial such as, but not limited to, a disorder or condition selected from the group consisting of: metabolic syndrome, glucose intolerance, hyperglycemia, dyslipidemia, diabetes mellitus type 1, diabetes mellitus type 2, hypertriglyceridemia, syndrome X, insulin resistance, impaired glucose tolerance (IGT), obesity, diabetic dyslipidemia, hyperlipidemia, arteriosclerosis, atherosclerosis, other cardiovascular diseases, hypertension, and complications resulting from or associated with diabetes including, but not limited to, neuropathy, retinopathy, nephropathy, and impaired wound healing. In an embodiment, the condition treated is type 2 diabetes.

The amorphous form of OAD2 dihydrochloride of the present invention may be administered at a dosage level such that the amount of OAD2 administered is between 1 mg and 1000 mg per day, or between 25 mg to 200 mg per day, or 25 to 75 mg per day, or 50 to 100 mg per day, or 75 to 125 mg per day, or 100 mg to 150 mg per day, or 125 to 175 mg per day, or 150 mg to 200 mg per day, or 75 mg to 150 mg per day. The dosage may be individualized by the clinician based on the specific clinical condition of the subject being treated.

It will be understood that the specific dosage level for any particular subject will depend upon a variety of factors including the activity of the specific compound employed, the age, body weight, general health, sex, diet, time of administration, route of administration, rate of excretion, drug combination and the severity of the particular disease undergoing therapy.

In additional embodiments, the amorphous form of OAD2 dihydrochloride salt of the present invention is used for the preparation of a medicament.

In another embodiment, the present invention provides the administration of the amorphous form of OAD2 dihydrochloride in combination therapy with one or more other active ingredients, for example other anti-diabetic drugs. The combination therapy may include the two or more active ingredients within a single pharmaceutical composition as well as the two or more active ingredients in two separate pharmaceutical compositions administered to the same subject simultaneously or at a time interval determined by a skilled artisan.

The invention will be further illustrated by combining the following specific examples. The following examples are used to explain the method of the invention and the core concept thereof, and for those skilled in the art, any possible change or substitution without departing from the inventive concept will fall within the protection scope of the invention. In the following examples, where the specific conditions of the experimental methods are not indicated, they are typically the conventional conditions, or are those recommended by the raw material or commodity manufactures; and the solvents without indicating the source are typically conventional solvents that are commercially available.

### EXAMPLES

### General Experimental Methods

Dynamic moisture adsorption (DVS) curves were collected on DVS Intrinsic of Surface Measurement Systems (SMS). The relative humidity at 25° C. was corrected with the deliquescent points of LiCl, Mg(NO₃)₂ and KCl. DVS test parameters are listed in Table 2 below.

TABLE 2
DVS Test Parameters

| DVS Test Parameters | Settings | |
|---|---|---|
| Temperature | 25° | C. |
| Sample | 10-25 | mg |
| Gas protection and flow speed | N2, 200 mL/minute | |
| dm/dt | 0.002%/min | |
| Min dm/dt balance time | 10 | min |
| Max balance time | 180 | min |

| | |
|---|---|
| RH range | 0% RH-95% RH |
| RH gradient | 10%(0% RH-90% RH, 90% RH-0% RH) |
| | 5%(90% RH-95% RH, 95% RH-90% RH) |

FIG. 1—XRPD Method Details—XRPD is performed using a Panalytical X-pert Pro MPD PW3040 Pro X-ray powder diffractometer. Analysis was performed from 2-40 degree 2 theta using the following conditions: X-Ray Tube: Cu-Kα (1.54059 Å), voltage: 45 kV, amperage: 40 mA, divergence slit, anti-scatter slit and detector slit set at 0.6 mm, receiving slit set at 0.1 mm, a step size of 0.02° and a scan speed of 3.3°/min.

FIG. 2—DSC Method Details—DSC is performed using a TA Instruments DSC Q2000 differential scanning calorimeter. The sample is placed into an aluminum DSC pan, and the weight accurately recorded. The pan is covered with a lid and then crimped. A weighed, crimped pan is placed on the reference side of the cell. The sample cell is equilibrated at −25° C. and heated under a nitrogen purge at a rate of 10° C./min, up to a final temperature of 225° C.

FIG. 3—TGA Method Details—TGA is performed using a TA instruments Q5000 IR thermo-gravimetric analyzer. The samples are analyzed using an open platinum pan. The sample is heated from 25° C. to 350° C. under a flow of nitrogen and a temperature rate of 10° C./min.

FIG. 4—FTIR Method Details—FTIR analysis of 1-3% dispersion in potassium bromide (KBr) is performed as described in the current USP under Chapter <197K>with a spectral resolution of 4 cm⁻¹.

FIG. 5—XRPD Method Details—XRPD is preformed using a d8 ADVANCE X-ray powder diffractometer. Scanning comprises the following steps: theta/2 theta. The scan range was 2-40 degree. The ray was a monochromatic Cu-Kα ray (lambda as 1.5406), voltage: 40 kV, and current: 40 mA.

FIG. 6—DSC Method Details—DSC is performed using a Mettler Toledo DSC 1 STARe System. The sample is subjected to the following conditions: pressure: 1013 MPa; the boosting rate: left: 20 mL/min, right: 100 mL/min; rate of temperature rise: 10.0° C./min; and temperature range: 0 to 300° C.

FIG. 7—TGA Method Details—TGA is performed using a Mettler Toledo TGA STARe System. The sample is subjected to the following conditions following purge with nitrogen: Rate of temperature rise: 20.0° C./min, and temperature range: 40-700° C.

### Example 1

Amorphous OAD2 dihydrochloride may be prepared by starting from the methyl ester of OAD2. The methyl ester of OAD2 was hydrolyzed using lithium hydroxide in tetrahydrofuran:methanol:water mixture. The reaction was neutralized with dilute hydrochloric acid and extracted with ethyl acetate. Concentration followed by dilution in ethyl acetate and treatment with saturated aqueous sodium carbonate afforded the solid sodium carboxylate. The collected sodium salt was slurried in acetone and treated with 4 N aqueous hydrochloric acid. The mixture was concentrated to afford the solid product, which is then washed with water and dried in vacuo to afford OAD2 dihydrochloride as an amorphous solid.

FIG. 1 illustrates a characteristic XRPD pattern of an amorphous form of OAD2 dihydrochloride sample obtained by using the preparation method of Example 1.

FIG. 2 illustrates a characteristic DSC of an amorphous form of OAD2 dihydrochloride sample obtained by using the preparation method of Example 1.

FIG. 3 illustrates a characteristic TGA of an amorphous form of OAD2 dihydrochloride sample obtained by using the preparation method of Example 1.

FIG. 4 illustrates a characteristic FTIR of an amorphous form of OAD2 dihydrochloride sample obtained by using the preparation method of Example 1. Table 1 lists individual peaks found in FIG. 4.

### Solubility

The solubility profile of OAD2 dihydrochloride salt as an amorphous solid from the method of Example 1 in aqueous solution at various pH levels is provided in Table 3 below.

TABLE 3
Solubility in Aqueous Solutions At Various pH's

| | Solubility |
|---|---|
| Solvent | (mg/mL) |
| 0.1 HCl | >51.0 |
| Buffer pH 5 | Not detectable |
| Buffer pH 7 | 0.09 |
| Buffer pH 9 | 0.05 |
| 0.1 NaOH | 0.017 |

### Example 2

Amorphous OAD2 dihydrochloride may also be prepared by starting from the methyl ester of OAD2. The methyl ester of OAD2 was diluted with 2-methyltetrahydrofuran, and the mixture was stirred at room temperature for 1 h. The mixture was cooled to 9.5° C., and 2N NaOH was added while maintaining a temperature below 20° C. The mixture was held between 15 and 23° C. for a day. The reaction mixture was cooled to about 5° C. and the pH of the mixture was adjusted to about 4 with 1 N HCl. The temperature of the reaction mixture was adjusted to about 10° C. and stirred for 2.5 h which resulted in formation of a slurry. The slurry was stirred at 15-20° C. for 1 h. The solids were collected by filtration and then washed with water and 2-methyltetrahydrofuran. The solid product was dried at 39° C. under reduced pressure to afford OAD2 monohydrochloride salt as a white solid.

The OAD2 monohydrochloride salt, 2-methyltetrahydrofuran and water were combined and heated to 55° C. and agitated at 55-65° C. for 2 h. The mixture was cooled to 25° C. over 1 h and stirred at room temperature for 1 h. The solids were filtered, washed with 2-methyltetrahydrofuran and dried at 39° C. under reduced pressure to afford OAD2 monohydrochloride salt as white solid.

OAD2 monohydrochloride salt was treated with acetic acid (1:2 by weight), and the mixture was stirred at room temperature for 30 min. 5N HCl was added over 10 min while maintaining a temperature below 25° C. The mixture was stirred at room temperature for 1 h. Water was added over 1 h while maintaining a temperature below 25° C. The mixture was stirred for at least 1.5 h to provide a clear solution which was transferred to a second reaction vessel along with acetic acid and water. 5N HCl was added while maintaining a temperature below 25° C. The mixture was stirred at 19.4-20.2° C. for 1 h. The resulting solids were collected by filtration and washed with 1 N HCl. The collected solids were treated with 1N HCl. The mixture was stirred at room temperature for 30 min, filtered, and the collected solid was washed with 1 N HCl. The collected solid was treated with 1 N HCl, and the mixture was stirred at room temperature for about 40 min, filtered, and the collected solid was washed with 1 N HCl. The solid product was dried at 40° C. under reduced pressure to afford an OAD2 dihydrochloride salt as an amorphous solid.

### Example 3

Amorphous OAD2 dihydrochloride may also be prepared by adding OAD2 (42.76 g, 0.05 mol) and acetic acid (86 mL) into a reaction flask, adding 4 mol/L hydrochloric acid solution (86 mL), and stirring until the system is completely dissolved. After the material is dissolved, purified water (850 mL) can be added followed by stirring for 1 hour, then adding 4 mol/L hydrochloric acid solution (250 mL), stirring for crystallization for 2 hours after the addition, filtering, and drying to obtain 46.4 g of the OAD2 dihydrochloride salt as an amorphous solid, wherein the yield is as follows: 99.8%, purity: 99.3 percent.

### Example 4

Amorphous OAD2 dihydrochloride may also be prepared by adding OAD2 (42.76 g, 0.05 mol) and tetrahydrofuran (86 mL) into a reaction flask, adding 4 mol/L hydrochloric acid solution (86 mL), and stirring until the system is completely dissolved. After the material is completely dissolved, purified water (850 mL) can be added followed by stirring for 1 hour, then adding 4 mol/L hydrochloric acid solution (250 mL), stirring for crystallization for 2 hours after the addition, filtering, and drying to obtain 45.9 g of the OAD2 dihydrochloride salt as an amorphous solid, wherein the yield is as follows: 98.7%, purity: 99.1 percent.

### Example 5

Amorphous OAD2 dihydrochloride may also be prepared by adding OAD2 (42.76 g, 0.05 mol) and acetone (86 mL) into a reaction flask, adding 4 mol/L hydrochloric acid solution (86 mL), and stirring until the system is completely dissolved. After the material is completely dissolved, purified water (850 mL) can be added followed by stirring for 1 hour, then adding 4 mol/L hydrochloric acid solution (250 mL), stirring for crystallization for 2 hours after the addition, filtering, and drying to obtain 46.0 g of the OAD2 dihydrochloride salt as an amorphous solid, wherein the yield is as follows: 98.9%, purity: 98.9 percent.

### Stability Study

A 30-day stability study at 60° C. was conducted on a sample of material obtained from the method of Example 3. The results are summarized in Table 4 below.

TABLE 4
30-day Stability Study at 60° C.

| | Day 0 | Day 5 | Day 10 | Day 30 |
|---|---|---|---|---|
| Appearance | Off-white | Off-white | Off-white | Pale-yellow |
| | solid | solid | solid | solid |
| Moisture | 1.6% | 2.7% | 2.4% | 2.5% |
| Content | | | | |
| Purity | 99.03% | 98.7% | 98.7% | 97.7% |
| Percentage | 97.1% | 99.1% | 97.5% | 97.4% |
| of external | | | | |
| standard | | | | |
| content | | | | |
| Solid form | Amorphous | Amorphous | Amorphous | Amorphous |

A 3-month stability study was conducted at 25° C.±2° C. and 60% relative humidity. The results are summarized in Table 5 below.

TABLE 5
3 Month Stability Study at 25° C. ± 2° C. and 60% RH

| Source of | | | | | |
|---|---|---|---|---|---|
| Sample | | Month 0 | Month 1 | Month 2 | Month 3 |
| Ex. 3 | Appearance | Off-white | Off-white | Off-white | Off-white |
| | | solid | solid | solid | solid |
| | Moisture Content | 1.7% | 3.1% | 3.4% | 3.7% |
| | Purity | 99.30% | 99.25% | 99.30% | 99.23% |
| | Percentage of | 98.1% | 99.0% | 98.7% | 99.8% |
| | external standard | | | | |
| | content | | | | |
| | Solid form | Amorphous | Amorphous | Amorphous | Amorphous |
| Ex. 4 | Appearance | Off-white | Off-white | Off-white | Off-white |
| | | solid | solid | solid | solid |
| | Moisture Content | 2.5% | 4.7% | 3.6% | 4.8% |
| | Purity | 98.7% | 98.6% | 98.6% | 98.5% |
| | Percentage of | 97.1% | 96.8% | 97.6% | 98.2% |
| | external standard | | | | |
| | content | | | | |
| | Solid form | Amorphous | Amorphous | Amorphous | Amorphous |
| Ex. 5 | Appearance | Off-white | Off-white | Off-white | Off-white |
| | | solid | solid | solid | solid |
| | Moisture Content | 1.6% | 3.3% | 4.1% | 4.5% |
| | Purity | 99.03% | 98.90% | 98.90% | 98.90% |
| | Percentage of | 97.1% | 97.2% | 97.8% | 98.7% |
| | external standard | | | | |
| | content | | | | |
| | Solid form | Amorphous | Amorphous | Amorphous | Amorphous |

### Dissolution Studies

Dissolution studies were conducted according to the four-part dissolution test method in the Chinese Pharmacopoeia (2015 edition) in the following solvents: methanol, acetic acid, ethanol, 0.1 mol/L HCl, acetonitrile, and 2-methyltetrahydrofuran. The dissolution study method involves weighing a fine powder (after grinding) of solid test product, attempting to dissolve in a proper volume of solvent by strongly shaking for 30 seconds every 5 minutes for 30 minutes, and recording observations after 30 minutes. If the solvent has no visually observable solute particles after 30 minutes, it is considered as completely dissolved. Results are summarized in Table 6 below.

TABLE 6
Solubility Study

| Source of | | Sample | Volume | | Volume of | |
|---|---|---|---|---|---|---|
| Sample | | weight | of solvent | | solvent | |
| (Example) | Solvent | (g) | (mL) | Dissolution | (mL) | Dissolution |
| 3 | Methanol | 1.00716 | 9.99 | Complete | — | — |
| 4 | | 1.00010 | 9.99 | Complete | — | — |
| 5 | | 1.00117 | 9.99 | Complete | — | — |
| 3 | Ethanol | 1.00206 | 9.99 | Incomplete | 29.99 | Complete |
| 4 | | 1.00127 | 9.99 | Incomplete | 29.99 | Complete |
| 5 | | 1.00214 | 9.99 | Incomplete | 29.99 | Complete |
| 3 | 0.1 mol/L HCl | 1.00691 | 9.99 | Incomplete | 29.99 | Complete |
| 4 | | 1.00136 | 9.99 | Incomplete | 29.99 | Complete |
| 5 | | 1.00135 | 9.99 | Incomplete | 29.99 | Complete |
| 3 | Acetonitrile | 0.01028 | 101 | Incomplete | — | — |
| 4 | | 0.01054 | 101 | Incomplete | — | — |
| 5 | | 0.01088 | 101 | Incomplete | — | — |
| 3 | 2-methyl | 0.01056 | 101 | Incomplete | — | — |
| 4 | tetrahydrofuran | 0.01062 | 101 | Incomplete | — | — |
| 5 | | 0.01038 | 101 | Incomplete | — | — |

It was found that the amorphous solid of OAD2 dihydrochloride salt completely dissolved in methanol, acetic acid, ethanol and 0.1 mol/L HCl in about 10 mL of solvent, and while it incompletely dissolved in acetonitrile and 2-methyltetrahydrofuran in about 101 mL of solvent.

FIG. 5 illustrates a characteristic XRPD pattern of a sample of an amorphous form of OAD2 dihydrochloride.

FIG. 6 illustrates a characteristic DSC of a sample of an amorphous form of OAD2 dihydrochloride.

FIG. 7 illustrates a characteristic TGA of a sample of an amorphous form of OAD2 dihydrochloride.

### Comparative Data

### Example C1: Preparation of Crystal Form B of OAD2 Hydrochloride

At 20° C., 1 g of OAD2 was added to 5 mL of 2-methyltetrahydrofuran, and then 1.4 mL of 1 mol/L hydrochloric acid solution was dropwise added. The mixture was stirred for 2 hours. XRPD was used to monitor whether a new crystal form was formed. After the completion of transformation and crystallization, crystal form B of OAD2 hydrochloride was obtained after suction filtration and drying at 50° C., with a salt formation ratio of 1:1. Crystal form B of the monohydrochloride of OAD2 may have an X-ray powder diffraction pattern comprising characteristic peaks at the following diffraction angles (2θ): 5.3±0.2°, 9.2±0.2°, 10.3±0.2°, 13.2±0.2°, and 14.8±0.2° . The crystal form B of monohydrochloride of OAD2 may also be characterized by an endothermic peak at 116° C. and/or 193° C. as determined by DSC.

The crystal form B of OAD2 hydrochloride was subject to tests to determine its pH solubility (Table 7), hygroscopicity (Table 8), and solid stability (Table 9). Details and results of these studies are provided below.

TABLE 7
Solubility of Crystal form B of
OAD2 hydrochloride At Various pH

| | Solubility (mg/mL) | | | | |
|---|---|---|---|---|---|
| | pH 1.0 | pH 2.0 | pH 3.0 | pH 5.0 | pH 7.0 |
| Crystal form B of | 0.381 | 0.017 | 0.002 | 0.001 | 0.109 |
| OAD2 hydrochloride | | | | | |

The crystal form B of OAD2 hydrochloride was subject to DVS test (25° C., 80% RH), and the results demonstrate that the sample showed no change of crystal form before and after the DVS test, and the sample had slight hygroscopicity, and the hygroscopicity was less than the hygroscopicity of the amorphous form of OAD2 dihydrochloride.

TABLE 8
Hygroscopicity Study Results

| | Moisture adsorption rate | Change of crystal form |
|---|---|---|
| Crystal form B of | 0.8% | No |
| OAD2 hydrochloride | | |
| OAD2 dihydrochloride | 8.3% | — |

The stability of crystal form B of OAD2 hydrochloride and the amorphous form of OAD2 dihydrochloride was tested under conditions of 40° C./100% RH for 1 week. Both samples showed no distinct decrease in purity.

TABLE 9
Stability Study Results at 40° C. and 100% RH for 7 days

| | HPLC (area %) | | Change of |
|---|---|---|---|
| Crystalline salt | Initiation | 40° C./100% RH/7 d | crystal form |
| Crystal form B of | 98.37 | 98.18 | No |
| OAD2 hydrochloride | | | |
| OAD2 | 98.91 | 97.51 | / |
| dihydrochloride | | | |

### Embodiments of the Invention:

Embodiment 1. An amorphous form of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid dihydrochloride salt (OAD2 dihydrochloride salt) characterized by an XRPD profile substantially as shown in FIG. 1.

Embodiment 2. An amorphous form of OAD2 dihydrochloride salt according to embodiment 1, further characterized by a DSC profile substantially as shown in FIG. 2.

Embodiment 3. An amorphous form of OAD2 dihydrochloride salt according to any one of the previous embodiments, characterized by a DSC profile having an endothermic peak between 85-105° C.

Embodiment 4. An amorphous form of OAD2 dihydrochloride salt according to any one of the previous embodiments, characterized by a TGA profile substantially as shown in FIG. 3.

Embodiment 5. An amorphous form of OAD2 dihydrochloride salt according to any one of the previous embodiments, characterized by a TGA profile having a weight loss of between 2-4% from 25 to 125° C.

Embodiment 6. An amorphous form of OAD2 dihydrochloride salt according to embodiment 5, further characterized by a TGA profile having a weight loss of between 22 to 28% from 190 to 310° C.

Embodiment 7. An amorphous form of OAD2 dihydrochloride salt according to any one of the previous embodiments, characterized by an IR profile substantially as shown in FIG. 4.

Embodiment 8. An amorphous form of OAD2 dihydrochloride salt according to any one of the previous embodiments, characterized by an IR profile having peaks at 1731±2 cm⁻¹, 1625±2 cm⁻¹, 1612±2 cm⁻¹, and 1509±2 cm⁻¹.

Embodiment 9. An amorphous form of OAD2 dihydrochloride salt according to any one of the previous embodiments, characterized by an IR profile having peaks at 1731±2 cm⁻¹, 1662±2 cm⁻¹, 1625±2 cm⁻¹, 1612±2 cm⁻¹, 1509±2 cm⁻¹, 1292±2 cm⁻¹, 764±2 cm⁻¹, and 668±2 cm⁻¹.

Embodiment 10. An amorphous form of OAD2 dihydrochloride salt according to any one of the previous embodiments, characterized by an FTIR profile having peaks at the wavenumbers (cm⁻¹) 3361, 3207, 2977, 2931, 2876, 1731, 1662, 1625, 1612, 1562, 1509, 1473, 1458, 1393, 1292, 1241, 1220, 1171, 1123, 1079, 1050, 1029, 1003, 877, 818, 764, 703, and 668.

Embodiment 11. A pharmaceutical composition comprising an amorphous form of OAD2 dihydrochloride salt according to any one of the previous embodiments and a pharmaceutically acceptable carrier.

Embodiment 12. A pharmaceutical composition of embodiment 11, comprising between 1 and 1000 mg of the amorphous form of OAD2 dihydrochloride salt.

Embodiment 13. A method of treating a condition comprising administering to a human in need thereof a therapeutically effective amount of an amorphous form of OAD2 dihydrochloride salt of any one of embodiments 1 to 10, wherein the condition is selected from the group consisting of metabolic syndrome, glucose intolerance, hyperglycemia, dyslipidemia, diabetes mellitus type 1, diabetes mellitus type 2, hypertriglyceridemia, syndrome X, insulin resistance, impaired glucose tolerance (IGT), obesity, diabetic dyslipidemia, hyperlipidemia, arteriosclerosis, atherosclerosis, other cardiovascular diseases, hypertension, and complications resulting from or associated with diabetes.

Embodiment 14. A method of embodiment 13, wherein the condition is type 2 diabetes.

Embodiment 15. A method of embodiment 14, wherein between 25 mg to 200 mg of OAD2 dihydrochloride salt is administered per day.

Embodiment 16. A method of preparing an amorphous form of OAD2 dihydrochloride salt according to any one of embodiments 1 to 10, wherein the method comprises:
i) adding an amount of OAD2 sodium salt or OAD2 monohydrochloride salt to a solvent or solvent system to form a mixture, and
ii) adding sufficient amount of an aqueous solution of HCl to the mixture to precipitate OAD2 dihydrochloride.

Embodiment 17. A method of preparing an amorphous form of OAD2 dihydrochloride salt according to any one of embodiments 1 to 10, wherein the method comprises:
i) adding OAD2 sodium salt to acetone and forming a slurry, and
ii) treating the slurry with an aqueous solution of HCl in an amount sufficient to form solid OAD2 dihydrochloride.

Embodiment 18. A method of preparing an amorphous form of OAD2 dihydrochloride salt according to any one of embodiments 1 to 10, wherein the method comprises:
i) dissolving OAD2 monohydrochloride salt in an aqueous solution of acetic acid and HCl, and
ii) adding sufficient amount of aqueous HCl to the aqueous solution to precipitate OAD2 dihydrochloride salt.

What is claimed is:

1. An amorphous form of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid dihydrochloride salt (OAD2 dihydrochloride salt) characterized by an XRPD profile substantially as shown in FIG. 1.

2. The amorphous form of OAD2 dihydrochloride salt according to claim 1, characterized by a DSC profile substantially as shown in FIG. 2.

3. The amorphous form of OAD2 dihydrochloride salt according to claim 1, characterized by a DSC profile having an endothermic peak between 85-105° C.

4. The amorphous form of OAD2 dihydrochloride salt according to claim 1, characterized by a TGA profile substantially as shown in FIG. 3.

5. The amorphous form of OAD2 dihydrochloride salt according to claim 1, characterized by a TGA profile having a weight loss of between 2-4% from 25 to 125° C.

6. The amorphous form of OAD2 dihydrochloride salt according to claim 5, further characterized by a TGA profile having a weight loss of between 22 to 28% from 190 to 310° C.

7. The amorphous form of OAD2 dihydrochloride salt according to claim 1, characterized by an IR profile substantially as shown in FIG. 4.

8. The amorphous form of OAD2 dihydrochloride salt according to claim 1, characterized by an IR profile having peaks at 1731±2 cm⁻¹, 1625±2 cm⁻¹, 1612±2 cm⁻¹, and 1509±2 cm⁻¹.

9. The amorphous form of OAD2 dihydrochloride salt according to claim 8, characterized by an IR profile having peaks at 1731±2 cm⁻¹, 1662±2 cm⁻¹, 1625±2 cm⁻¹, 1612±2 cm⁻¹, 1509±2 cm⁻¹, 1292±2 cm⁻¹, 764±2 cm⁻¹, and 668±2 cm⁻¹.

10. The amorphous form of OAD2 dihydrochloride salt according to claim 1, characterized by an FTIR profile having peaks at the wavenumbers (cm⁻¹) 3361, 3207, 2977, 2931, 2876, 1731, 1662, 1625, 1612, 1562, 1509, 1473, 1458, 1393, 1292, 1241, 1220, 1171, 1123, 1079, 1050, 1029, 1003, 877, 818, 764, 703, and 668.

11. A pharmaceutical composition comprising an amorphous form of OAD2 dihydrochloride salt according to any one of claims 1 to 10 and a pharmaceutically acceptable carrier.

12. The pharmaceutical composition of claim 11, comprising between 1 and 1000 mg of the amorphous form of OAD2 dihydrochloride salt.

13. A method of treating a condition comprising administering to a human in need thereof a therapeutically effective amount of an amorphous form of OAD2 dihydrochloride salt of any one of claims 1 to 10, wherein the condition is selected from the group consisting of metabolic syndrome, glucose intolerance, hyperglycemia, dyslipidemia, diabetes mellitus type 1, diabetes mellitus type 2, hypertriglyceridemia, syndrome X, insulin resistance, impaired glucose tolerance (IGT), obesity, diabetic dyslipidemia, hyperlipidemia, arteriosclerosis, atherosclerosis, other cardiovascular diseases, hypertension, and complications resulting from or associated with diabetes.

14. The method of claim 13, wherein the condition is type 2 diabetes.

15. The method of claim 14, wherein between 25 mg to 200 mg of OAD2 dihydrochloride salt is administered per day.

16. A method of preparing an amorphous form of OAD2 dihydrochloride salt according to any one of claims 1 to 10, wherein the method comprises:
i) adding an amount of OAD2 sodium salt or OAD2 monohydrochloride salt to a solvent or solvent system to form a mixture, and
ii) adding sufficient amount of an aqueous solution of HCl to the mixture to precipitate OAD2 dihydrochloride.

17. A method of preparing an amorphous form of OAD2 dihydrochloride salt according to any one of claims 1 to 10, wherein the method comprises:
i) adding OAD2 sodium salt to acetone and forming a slurry, and
ii) treating the slurry with an aqueous solution of HCl in an amount sufficient to form solid OAD2 dihydrochloride.

18. A method of preparing an amorphous form of OAD2 dihydrochloride salt according to any one of claims 1 to 10, wherein the method comprises:
i) dissolving OAD2 monohydrochloride salt in an aqueous solution of acetic acid and HCl, and
ii) adding sufficient amount of aqueous HCl to the aqueous solution to precipitate OAD2 dihydrochloride salt.
